(19) United States
(12) Patent Application Publication
(10) Pub. No.: US 2024/0207106 A1
(43) Pub. Date: Jun. 27, 2024

(54) ABSORBENT ARTICLE WITH ABSORBENT WAIST BARRIER

(71) Applicant: Irving Consumer Products Limited, Dieppe (CA)

(72) Inventors: Frank S. Glaug, Chester Springs, PA (US); Jason Harry Ned Green, Irishtown (CA); Alaa Sadek, Moncton (CA)

(73) Assignee: Irving Consumer Products Limited, Dieppe (CA)

(21) Appl. No.: 18/392,690

(22) Filed: Dec. 21, 2023

Related U.S. Application Data
(60) Provisional application No. 63/434,512, filed on Dec. 22, 2022.

(51) Int. Cl. A61F 13/494; A61F 13/15; A61F 13/49

(52) U.S. Cl. CPC A61F 13/49466; A61F 13/15707; A61F 13/49011; A61F 2013/49093

(57) ABSTRACT

The present invention relates generally to absorbent articles, and, more particularly, to absorbent articles that provide improvements in the containment of bodily fluid secretions. The fluid containment improvements are the result of adding one or more absorbent barriers at one or both of the longitudinally distal ends of the absorbent core. Adding absorbent barrier(s) that partially overlap the absorbent core to absorbent articles may decrease fluid leakage at the front, back, or both waist areas of the user. The absorbent barrier(s) may improve the comfort of the user during nap time, overnight, or whenever the user is lying on their back or stomach. The absorbent barrier may be formed of an airlaid material.

### CROSS-REFERENCE TO RELATED APPLICATIONS

This application claims the benefit of U.S. Provisional Application No. 63/434,512 filed on Dec. 22, 2022, which is incorporated by reference herein in its entirety.

### FIELD

The described embodiments relate generally to absorbent articles, and, more particularly, to absorbent articles having an absorbent waist barrier.

### BACKGROUND

The following is not an admission that anything discussed below is part of the prior art or part of the common general knowledge of a person skilled in the art.

There are several types of commercially available products for the absorption of bodily fluids. Such absorbent products are available in different types, designs, and dimensions, each one having one or more unique features. For example, training pants, baby diapers, adult diapers, adult pants, and incontinence guards are products designed for the containment of urine and excrement. There are other types of absorbent articles, such as feminine hygiene products (e.g., heavy and light incontinence pads, panty liners, etc.) that are designed to contain and absorb urine and/or menses by female wearers.

Absorbent articles typically include a topsheet facing the body of the wearer, a backsheet facing the garment of the wearer and an absorbent core located between the topsheet and backsheet. In some cases, an acquisition layer may be provided between the topsheet and the absorbent core. An acquisition layer may help in the fast absorption and distribution of bodily fluids from the topsheet to the absorbent core. In addition, barrier leg cuffs may be provided at each side of the absorbent core in the longitudinal direction of the product, to help prevent side leakage of the bodily fluids within the crotch and leg regions of the body.

While these types of absorbent articles may collect body fluid discharge as intended, many of these products tend to leak when exposed to higher fluid volumes, longer periods of wear, and increased stress conditions when the user is active. Further, the products known in the art are prone to leakage when worn by a wearer while they are sleeping. This leakage may lead to frequent changing of the user's outer garments, bed sheets, baby seat covers, etc. In addition to the nuisance of cleaning-up and washing clothes, there is always the embarrassment to both the user and caretaker of soiled clothing and surroundings.

Current commercially available absorbent articles are generally insufficient in providing an effective fluid barrier that substantially prevents fluid leakage, especially when the product is relatively saturated with fluid and is worn for long periods of time, such as overnight use. There are current commercially available absorbent products that contain waist barriers, containment pouches, or waistbands that help reduce leakage at the either front or back end of the product or both. Unfortunately, these executions offer limited leakage protection at the front or back waist areas of the product when the absorbent core becomes relatively saturated and/or if there is a gap between the product and user's skin.

### SUMMARY

The following introduction is provided to introduce the reader to the more detailed discussion to follow. The introduction is not intended to limit or define any claimed or as yet unclaimed invention. One or more inventions may reside in any combination or sub-combination of the elements or process steps disclosed in any part of this document including its claims and figures.

In accordance with one aspect of this disclosure, which may be used alone or in combination with any other aspect, there is provided an absorbent article comprising:
a front waist region having front waist edge, a rear waist region having a rear waist edge, and a crotch region extending longitudinally between the front waist region and the rear waist region;
a liquid pervious topsheet on a skin facing side of the absorbent article;
a liquid impervious backsheet on a garment facing side of the absorbent article;
an absorbent core positioned between the topsheet and the backsheet in a thickness direction, the absorbent core having a front core edge a rear core edge and a pair of side core edges extending between the front core edge and the rear core edge; and
an absorbent barrier, the absorbent barrier having a front barrier edge, a rear barrier edge, and a pair of side barrier edges extending between the front barrier edge and the rear barrier edge,
   the front barrier edge positioned longitudinally forward the front core edge,
   the rear barrier edge positioned longitudinally rearward the front core edge, and
   the rear barrier edge positioned between the absorbent core and the topsheet in the thickness direction.

In any embodiment, the front barrier edge may be positioned longitudinally between the front core edge and the front waist edge, and the rear barrier edge may be positioned longitudinally between the front core edge and the rear core edge.

In any embodiment, the front core edge may be longitudinally spaced from the front waist edge by a first distance, the rear core edge may be longitudinally spaced from the rear waist edge by a second distance, and the first distance may be greater than the second distance.

In any embodiment, the first distance may be at least 20 mm greater than the second distance.

In any embodiment, absorbent barrier may directly abut the absorbent core.

In any embodiment, the absorbent barrier may be secured to the topsheet and the absorbent core may be secured to the backsheet.

In any embodiment, the front barrier edge and the rear barrier edge may be longitudinally equally spaced from the front core edge.

In any embodiment, the front barrier edge may be longitudinally spaced between 20 mm and 30 mm from front waist edge.

In any embodiment, the absorbent article may further comprise a first longitudinally extending barrier leg cuff and a second longitudinally extending barrier leg cuff, the first barrier leg cuff and the second barrier leg cuff may be laterally spaced apart by a third distance, wherein the absorbent barrier may have a lateral width; and the lateral width of the absorbent barrier may be greater than the third distance.

In any embodiment, the absorbent article may further comprise an acquisition layer positioned between the topsheet and the absorbent core in the thickness direction, the acquisition layer having a front acquisition edge and a rear acquisition edge, each of the front acquisition edge and the rear acquisition edge may be positioned longitudinally rearward rear barrier edge.

In any embodiment, the absorbent barrier may be formed from an airlaid material.

In any embodiment, the absorbent article may further comprise a second absorbent barrier, the second absorbent barrier having a front barrier edge, a rear barrier edge, and a pair of side barrier edges, the front barrier edge of the second absorbent barrier may be positioned longitudinally forward the rear core edge, the rear barrier edge of the second absorbent barrier may be positioned longitudinally rearward the rear core edge, and the front barrier edge of the second absorbent barrier may be positioned between the absorbent core and the topsheet in the thickness direction.

In any embodiment, the front barrier edge of the second absorbent barrier may be positioned longitudinally between the rear core edge and the front core edge, and the rear barrier edge of the second absorbent barrier may be positioned longitudinally between the rear core edge and the rear waist edge.

In any embodiment, the front barrier edge of the second absorbent barrier and the rear barrier edge of the second absorbent barrier may be longitudinally equally spaced from the rear core edge.

In any embodiment, the rear barrier edge of the absorbent barrier and the front barrier edge of the second absorbent barrier may be longitudinally spaced apart.

In accordance with another aspect of this disclosure, which may be used alone or in combination with any other aspect, there is provided an absorbent article comprising:
a front waist region having front waist edge, a rear waist region having a rear waist edge, and a crotch region extending longitudinally between the front waist region and the rear waist region,
a liquid pervious topsheet on a skin facing side of the absorbent article;
a liquid impervious backsheet on a garment facing side of the absorbent article;
an absorbent core positioned between the topsheet and the backsheet in a thickness direction, the absorbent core having a front core edge a rear core edge and a pair of side core edges extending between the front core edge and the rear core edge; and
an absorbent barrier, the absorbent barrier having a front barrier edge, a rear barrier edge, and a pair of side barrier edges extending between the front barrier edge and the rear barrier edge,
   the front barrier edge positioned longitudinally forward the rear core edge,
   the rear barrier edge positioned longitudinally rearward the rear core edge, and
   the front barrier edge positioned between the absorbent core and the topsheet in the thickness direction.

In any embodiment, the rear barrier edge may be positioned longitudinally between the rear core edge and the rear waist edge, and the front barrier edge may be positioned longitudinally between the front core edge and the rear core edge.

In any embodiment, the front barrier edge and the rear barrier edge may be longitudinally equally spaced from the rear core edge.

In accordance with another aspect of this disclosure, which may be used alone or in combination with any other aspect, there is provided a method of manufacturing an absorbent article comprising:
providing a topsheet, a backsheet, an absorbent core, and an absorbent barrier;
securing the absorbent barrier to the topsheet; securing the absorbent core to the backsheet;
positioning the absorbent barrier so that a first portion of the absorbent barrier overlaps with the absorbent core in a thickness direction and a second portion of the absorbent barrier does not overlap with the absorbent core in the thickness direction; and
securing the topsheet to the backsheet.

In any embodiment, the method may further comprise providing an elastic waist band and securing the elastic waistband to at least one of the topsheet and the backsheet.

In any embodiment, the method may further comprise providing a second absorbent barrier; securing the second absorbent barrier to the topsheet; and positioning the second absorbent barrier so that a first portion of the second absorbent barrier overlaps with the absorbent core in a thickness direction and a second portion of the second absorbent barrier does not overlap with the absorbent core in the thickness direction; wherein the absorbent core has a first distal end and a second distal end and the absorbent barrier is positioned at the first distal end of the absorbent core and the second absorbent barrier is positioned at the second distal end of the absorbent core.

In any embodiment, the method may further comprise providing a second elastic waistband and securing the second elastic waist band to at least one of the topsheet and the backsheet, wherein the absorbent article has a front waist region and a rear waist region and the elastic waist band is secured to the at least one of the topsheet and the backsheet in the front waist region and the second elastic waist band is secured to the at least one of the topsheet and the backsheet in the rear waist region.

In any embodiment, the method may further comprise directly abutting the absorbent barrier with the absorbent core.

These and other aspects and features of various embodiments will be described in greater detail below.

### BRIEF DESCRIPTION OF THE DRAWINGS

For a better understanding of the described embodiments and to show more clearly how they may be carried into effect, reference will now be made, by way of example, to the accompanying drawings in which:

FIG. 1A shows a top view of a first embodiment of an absorbent article;

FIG. 1B shows a cross-sectional view of the absorbent article of FIG. 1A, taken along line 1B-1B;

FIG. 2A shows a top view of a second embodiment of an absorbent article;

FIG. 2B shows a cross-sectional view of the absorbent article of FIG. 2A, taken along line 2B-2B;

FIG. 3A shows a top view of a third embodiment of an absorbent article;

FIG. 3B shows a cross-sectional view of the absorbent article of FIG. 3A, taken along line 3B-3B;

FIG. 4A shows a top view of a fourth embodiment of an absorbent article;

FIG. 4B shows a cross-sectional view of the absorbent article of FIG. 3A, taken along line 4B-4B; and

FIG. 4C shows a cross-sectional view of the absorbent article of FIG. 4A, taken along line 4C-4C.

The drawings, described below, are provided for purposes of illustration, and not of limitation, of the aspects and features of various examples of embodiments described herein. For simplicity and clarity of illustration, elements shown in the drawings have not necessarily been drawn to scale. The dimensions of some of the elements may be exaggerated relative to other elements for clarity. It will be appreciated that for simplicity and clarity of illustration, where considered appropriate, reference numerals may be repeated among the drawings to indicate corresponding or analogous elements or steps.

### DESCRIPTION OF EXAMPLE EMBODIMENTS

Various apparatuses and methods are described below to provide an example of an embodiment of each claimed invention. No embodiment described below limits any claimed invention and any claimed invention may cover apparatuses and methods that differ from those described below. The claimed inventions are not limited to apparatuses and methods having all of the features of any one apparatus or method described below or to features common to multiple or all of the apparatuses or methods described below. It is possible that an apparatus or method described below is not an embodiment of any claimed invention. Any invention disclosed in an apparatus or method described below that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

The terms “an embodiment,” “embodiment,” “embodiments,” “the embodiment,” “the embodiments,” “one or more embodiments,” “some embodiments,” and “one embodiment” mean “one or more (but not all) embodiments of the present invention(s),” unless expressly specified otherwise.

The terms “including,” “comprising” and variations thereof mean “including but not limited to,” unless expressly specified otherwise. A listing of items does not imply that any or all of the items are mutually exclusive, unless expressly specified otherwise. The terms “a,” “an” and “the” mean “one or more,” unless expressly specified otherwise.

It should be noted that terms of degree such as “substantially”, “about”, and “approximately” when used herein mean a reasonable amount of deviation of the modified term such that the end result is not significantly changed. These terms of degree should be construed as including a deviation of the modified term if this deviation would not negate the meaning of the term it modifies.

As used herein and in the claims, two or more parts are said to be “coupled”, “connected”, “attached”, or “fastened” (which may all be used interchangeably) where the parts are joined or operate together either directly or indirectly (i.e., through one or more intermediate parts), so long as a link occurs. As used herein and in the claims, two or more parts are said to be “directly coupled”, “directly connected”, “directly attached”, or “directly fastened” where the parts are connected in physical contact with each other. None of the terms “coupled”, “connected”, “attached”, and “fastened” distinguish the manner in which two or more parts are joined together. For example, two or more parts may be “coupled”, “connected”, “attached” or “fastened” by bonding them together with an ultrasonic or heat bond or other technique that does not require a bonding agent, with a bonding agent such as an adhesive, through mechanical bonding, with a mechanical fastener, or in any other manner.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

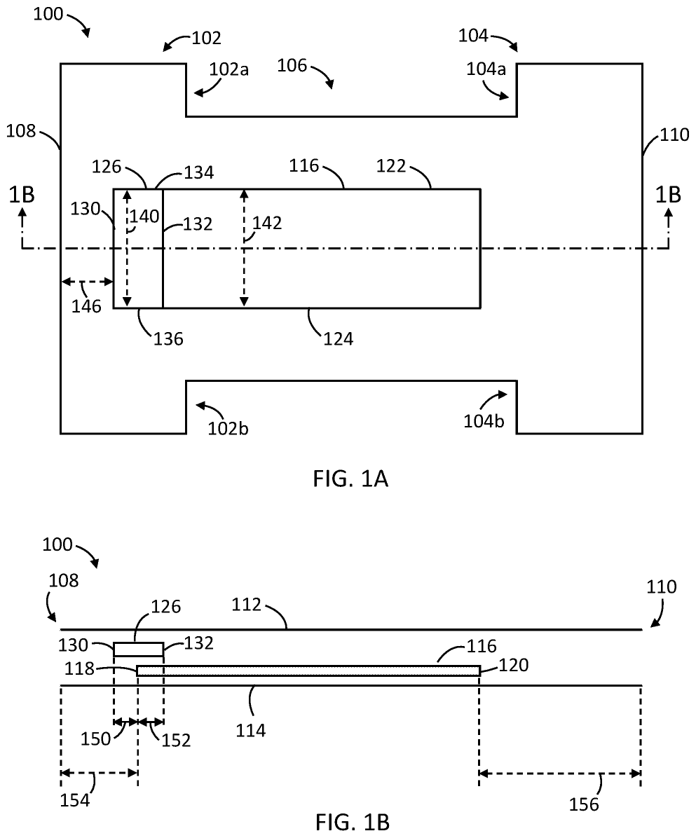

Referring first to FIGS. 1A and 1B, shown therein is an example embodiment of an absorbent article 100. As shown, the absorbent article 100 may include a front waist region 102, a rear waist region 104, and a crotch region 106 that extends between the front waist region 102 and the rear waist region 104. In the example shown, the front and rear waist regions 102, 104 include respective right and left side panels 102a, 102b, 104a, 104b. It will be appreciated that the distal ends of the right side panels 102a, 104a and the distal ends of the left side panels 102b, 104b may be joined to form a waist opening and a pair of leg openings. More specifically, a front waist edge 108 of the front waist region 102 and a rear waist edge 110 of the rear waist region 104 may together form the waist opening. The right and left side panels 102a, 102b, 104a, 104b may be rigidly coupled (e.g., welded) together or may include refastenable joining members (e.g., hooks and loops). In other embodiments, at least one of the front and rear waist regions 102, 104 may not include side panels.

Still referring to FIGS. 1A and 1B, the absorbent article 100 shown has a topsheet 112 and a backsheet 114. As shown in FIG. 1B, the topsheet 112 is positioned on the skin facing side of the absorbent article 100 and the backsheet 114 is positioned on the garment facing side of the absorbent article 100.

The topsheet 112 may be formed of any material known in the art suitable for (a) contacting the skin of the wearer; and (b) receiving an insult of liquid (e.g., urine, menses, etc.) and/or excrement from a wearer of the absorbent article 100. For example, the topsheet 112 may be manufactured from woven and/or nonwoven materials (e.g., a nonwoven web of fibers); spunbond webs, spunlace webs, carded webs, polymeric materials such as apertured formed thermoplastic films, apertured plastic films, and hydroformed thermoplastic films; porous foams; reticulated foams; reticulated thermoplastic films; and thermoplastic scrims. The topsheet 112 may or may not be treated with, for example, a surfactant. When the topsheet 112 comprises a nonwoven web, the web may be spunbonded, carded, wet-laid, melt-blown, hydroentangled, combinations of the above, or the like. In the example illustrated in FIG. 1B, the topsheet 112 is formed from a single layer. In other embodiments, the topsheet 112 may include multiple layers.

The backsheet 114 may be formed of any material known in the art suitable for preventing the insult received by the absorbent article 100 from wetting a garment or surface external to the absorbent article 100 (e.g., bedsheets). For example, the backsheet 114 may be manufactured from woven and/or nonwoven materials, polymeric films such as thermoplastic films of polyethylene or polypropylene, composite materials such as a film-coated nonwoven material, or sustainable materials such as bamboo, cotton, and viscose. Optionally, the backsheet 114 may permit vapors to escape from the absorbent article 100 (i.e., the backsheet 114 may be breathable) while still preventing exudates from passing through the backsheet 114. In the example illustrated in FIG. 1B, the backsheet 114 is formed of a single layer. In other embodiments, the backsheet 114 may include multiple layers. For example, the backsheet 114 may include a layer of a liquid-impervious material (e.g., a poly-film) and a non-woven layer.

Still referring to FIGS. 1A and 1B, the absorbent article 100 shown includes an absorbent core 116 positioned between the topsheet 112 and the backsheet 114 in the thickness direction. The absorbent core 116 may be of any shape and size known in the art. In the example illustrated, the absorbent core 116 is generally rectangular in shape and has a front core edge 118, a rear core edge 120, and a pair of side core edges 122, 124 extending between the front core edge 118 and the rear core edge 120.

The absorbent core 116 may be formed of any material known in the art suitable for absorbing bodily fluids. In some embodiments, the absorbent core 116 may include a plurality of absorbent materials. It will be appreciated that the absorbent core 116 may include pulp, SAP, or a blend of pulp and SAP. In the example illustrated in FIGS. 1A and 1B, the absorbent core 116 includes a single layer containing a blend of pulp and SAP. It will be appreciated that the absorbent material in the absorbent core 116 may be a single pulp material or a plurality of pulp materials. For example, the absorbent material may include one or more of the following: rayon fibers, cotton fibers, bamboo fibers, soft pine, eucalyptus fibers, superabsorbent polymer particles, superabsorbent polymer fibers, peat moss, cross-linked cellulose fibers, cellulose acetate, polypropylene tow, polymer fibers, surfactant treated nonwovens, binder fibers, linen fibers, hemp fibers, ramie fibers, jute fibers, miscanthus pulp fibers, natural cellulose fibers, sponges, absorbent foams, etc. In some embodiments, the absorbent core 116 may include a core wrap (not shown) that encases at least a portion of the absorbent material of the absorbent core 116.

As shown in FIGS. 1A and 1B, the absorbent article 100 may also include an absorbent barrier 126 positioned between the topsheet 112 and the backsheet 114 in the thickness direction. It may be desirable to include an absorbent barrier 126 in the absorbent article 100 to reduce the likeliness of an insult from leaking out from the absorbent article 100.

As described above, it has been found that absorbent articles known in the art are prone to leakage. Absorbent articles may leak when the absorbent core thereof becomes overwhelmed by a high volume of fluid emitted thereon in a short period of time. This leakage has been determined to be more likely when the wearer of the article is sleeping or laying down (on side, back, or front) due to inadequate absorbent and/or barrier members for limiting leakage of fluids out the waist opening.

Testing absorbent articles known in the art on mannequins has demonstrated that known commercial products are limited in leakage protection at the front and back waist areas, especially when the absorbent core becomes saturated. For instance, when the user is laying on either their belly or back, the fluid flows down the absorbent core to the lowest point in the absorbent article. The lowest point can be either in the front section of the absorbent article, when laying on their belly, or in the rear section of the absorbent article, when laying on their back. The fluid can flow down to these sections and over saturate the absorbent core. Saturation of the absorbent core happens especially when the user is laying on their back or stomach for long periods of time, such as overnight. Urine may gush down to the back or front end of the product, due to gravity and the position of the user, similar to a waterfall. When the absorbent core is saturated at the front or back section, the fluid can easily leak out at either the front waist edge or back waist edge of the absorbent article.

Typical previously known waist barriers, containment pouches and elastic waistbands offer limited protection to prevent the urine from flowing out of the product, thereby causing leakage. They are flattened out by both the weight of the user and the tension applied to the user when the article is worn. As the absorbent core gets saturated, the superabsorbent polymer swells and the urine eventually flows along the top surface. The swelling effect of the core allows it to rise above the commercial waist barrier executions and the urine simply flows over the top and leaks out the product. Additionally, the tightness of the waistband against the skin, can cause red marking. Red markings may occur because absorbent products are usually applied taut to the body to reduce drooping and the tension at the waist may be increased when the user moves from standing to sitting positions.

Another deficiency with typical previously known waist barriers is that they do not retain any urine. There are many instances where urine flows outside of the absorbent core and becomes “free fluid”. This “free fluid” can leak out of the product when pressure is applied to the product or when the user moves.

Accordingly, it may be desirable to provide a more efficient absorbent barrier that may improve leakage protection, primarily during overnight use, along the front and/or back waist regions 102, 104 of the absorbent article 100.

The absorbent barrier 126 may be of any shape and size suitable (a) for absorbing at least a portion of an insult not captured by the absorbent core 116 and (b) not significantly reducing the comfort of the absorbent article 100. Further, the absorbent barrier 126 may be made of any material known in the art. For example, like the absorbent core 116, the absorbent barrier may include a plurality of absorbent materials such as pulp, SAP, or a blend of pulp and SAP. It will be appreciated that the absorbent material in the absorbent barrier may be a single pulp material or a plurality of pulp materials. For example, the absorbent material may include one or more of the following: rayon fibers, cotton fibers, bamboo fibers, soft pine, eucalyptus fibers, superabsorbent polymer particles, superabsorbent polymer fibers, peat moss, cross-linked cellulose fibers, cellulose acetate, polypropylene tow, polymer fibers, surfactant treated nonwovens, binder fibers, linen fibers, hemp fibers, ramie fibers, jute fibers, miscanthus pulp fibers, natural cellulose fibers, sponges, absorbent foams, etc.

Optionally, the absorbent barrier 126 may be an airlaid material. Specifically, the absorbent barrier 126 may be a multi bonded airlaid SAP produced by Glatfelter having the properties outlined in Table 1, below (Product: MM390.SMT.CSN00.V00.J.NoCF (2000001409)).

TABLE 1

| Property | Value/Unit | | Test Method |
|---|---|---|---|
| Basis Weight | 390 | gm/² | NWSP 130.1.R0 (15) |
| Thickness (2.0 kPa) | 2.03 | mm | NWSP 120.6R0 (15) |
| Density | 0.192 | g/cm³ | Calculated |
| Tensile Strength (dry) MD | 52 N/50 mm | | NWSP 110.4.R0 (15) |
| Absorption (0.9% saline) | 21 | g/g | NWSP 10.1.R0 (15) |
| Retention (0.9% saline) | 14 | g/g | NWSP 241.0.R2 (15) |
| Liquid Strike Through (0.9% | <3 | sec | NWSP 70.3.R0 (15) |
| saline) | | | |

Referring to FIGS. 1A and 1B, as shown, a first portion of the absorbent barrier 126 may overlap with a portion of the absorbent core 116 and a second portion of the absorbent barrier 126 may extend laterally beyond the absorbent core 116. It has been determined that partially overlapping the absorbent barrier 126 with the absorbent core 116 may allow for the absorbent barrier 126 to limit leakage of urine and other bodily fluid out from the waist edges 108, 110 of the absorbent article 100.

More specifically, as shown in FIG. 1B, the absorbent barrier 126 may be placed in the absorbent article 100 such that approximately half of the absorbent barrier 126 overlaps with the absorbent core 116 and approximately half of the absorbent barrier 126 does not overlap with the absorbent core 116 (i.e., a portion of the absorbent barrier 126 may extend beyond the absorbent core 116 and onto the backsheet 114). It may be desirable to position the absorbent barrier 126 in this way to provide double protection against fluid leakage at the waist region of the absorbent article 100. That is, positioning the absorbent barrier 126 with approximately half the surface area thereof overlapping the core 116 and approximately half of the surface area thereof not overlapping the core (1) the absorbent barrier 126 may cover the end of the absorbent core 116 and extend onto the backsheet 114 which may act as a barrier to stop any fluid that wicks through the absorbent core 114 from escaping from the absorbent article; and (2) the absorbent barrier 126 may cover a portion of the upper surface of the absorbent core 116 which may act as a barrier to stop fluid that flows along the surface of the topsheet 112, when the absorbent core 114 is saturated and the fluid can no longer be absorbed by the absorbent core 114.

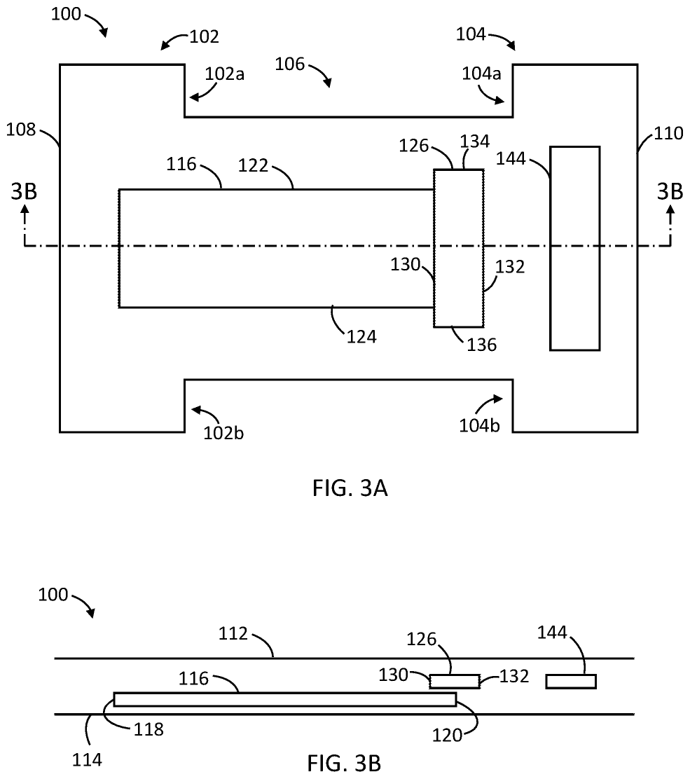

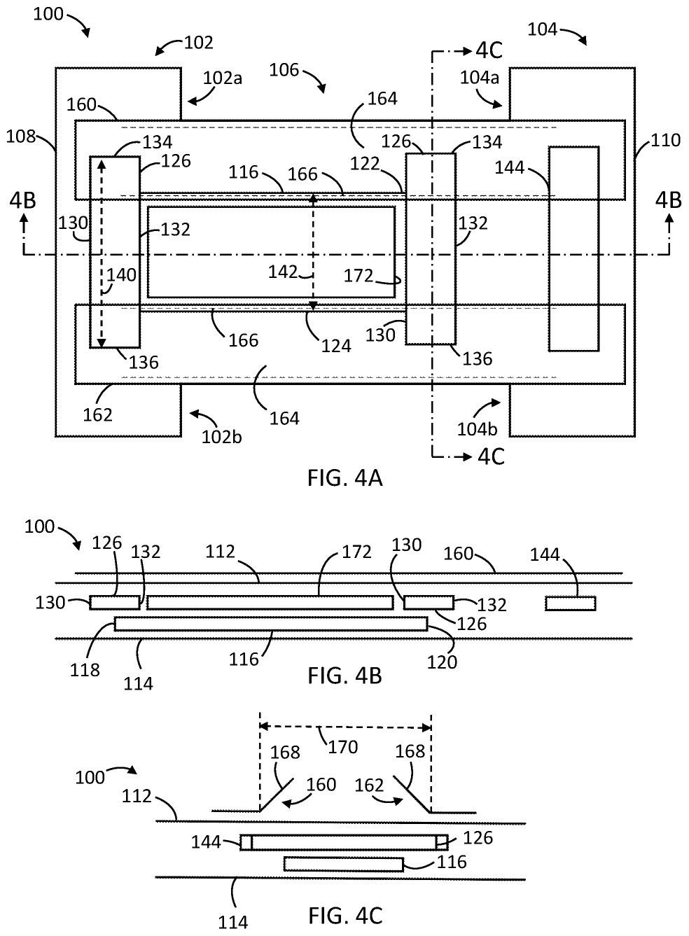

While the example illustrated in FIGS. 1A and 1B shows an absorbent article 100 having an absorbent barrier 126 positioned only at the front core edge 118, it is to be understood that an absorbent article 100 may include an absorbent barrier 126 positioned only at the rear core edge 120 (see, e.g., FIGS. 3A and 3B) or include an absorbent barrier 126 positioned at each of the front core edge 118 and the rear core edge 120 (see, e.g., FIGS. 4A to 4C).

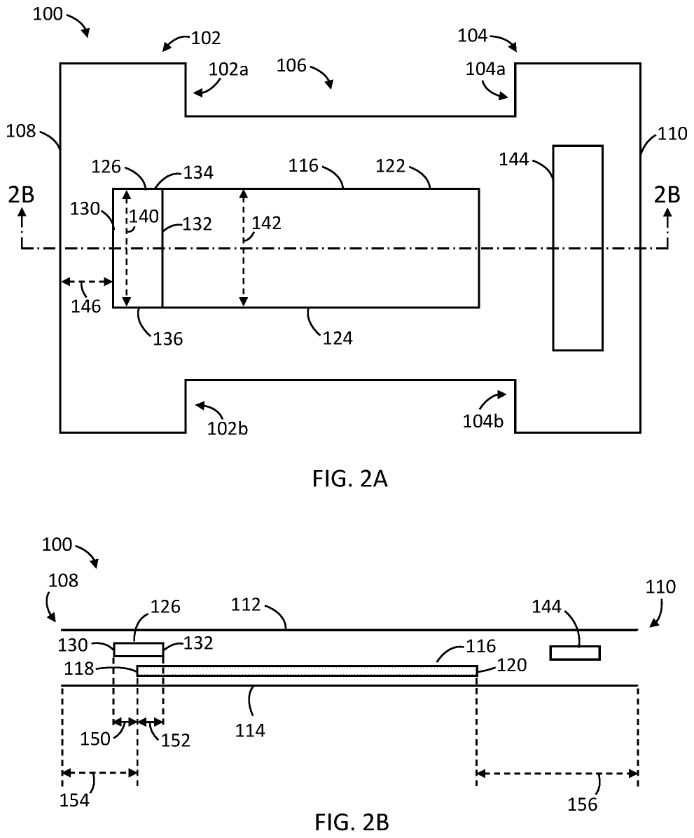

In the description of the absorbent barrier 126 that follows, reference is primarily made to the embodiment of the absorbent article 100 shown in FIGS. 1A and 1B. However, it is to be understood that the description of the absorbent barrier 126 of FIGS. 1A and 1B may be applied to that of FIGS. 2A to 4C.

As shown in FIG. 1B, the absorbent barrier 126 may include a front barrier edge 130, a rear barrier edge 132, and a pair of barrier side edges 134, 136. In the example shown, the absorbent barrier 126 is generally rectangular in shape but it is to be understood that the absorbent barrier 126 may be of any shape known in the art.

As previously described, the absorbent barrier 126 may be positioned so that a first portion of the absorbent barrier 126 overlaps the absorbent core 116 and a second portion of the absorbent barrier 126 does not overlap the absorbent core 116. The portion of the absorbent barrier 126 that overlaps the absorbent core 116 may make up any percentage of the total surface area of the absorbent barrier 126. Optionally, as shown, the portion of the absorbent barrier 126 that overlaps the core may comprise 50% of the total surface of the absorbent barrier 126.

In the example illustrated in FIG. 1A, the absorbent barrier 126 has a lateral width 140 which is equal to the lateral width 142 of the absorbent core 116. In other embodiments, the lateral width 140 of the absorbent barrier 126 may be less than that of the absorbent core 116 or greater than that of the absorbent core 116 (see, e.g., FIGS. 3A to 4C).

In the example illustrated in FIGS. 1A and 1B, the absorbent barrier 126 is positioned so that the front barrier edge 130 is positioned longitudinally between the front core edge 118 and the front waist edge 108 and the rear barrier edge 132 is positioned longitudinally between the front core edge 118 and the rear core edge 120. In other embodiments, the front barrier edge 130 may extend to the front waist edge 108 (i.e., be coterminous with the front waist edge 108 in the thickness direction). While the front barrier edge 130 may extend to the front waist edge 108, it may be desirable for the front barrier edge 130 to be offset therefrom, as shown, so that a seal between the topsheet 112 and the backsheet 114 can be formed longitudinally outboard of the absorbent barrier 126. It has been found that sealing the topsheet 112 to the backsheet 114 longitudinally outboard of the absorbent barrier 126 may reduce the likeliness of bodily fluids from penetrating through the absorbent barrier 126 and escaping the absorbent article 100 through the waist edge(s) 108, 110.

That is, for example, and as shown in FIG. 1B, the front barrier edge 130 may be longitudinally spaced from the front waist edge 108 by a distance 146. Optionally, the distance 146 may be between 20 mm and 30 mm. It has been determined that longitudinally spacing the front barrier edge 130 of the absorbent barrier 126 from the front waist edge 108 by 20 mm to 30 mm (a) allows for the topsheet 112 and the backsheet 114 to be sealed outboard of the absorbent barrier 126; and (b) provides a zone for waist elastics (not shown) to be positioned outboard of the absorbent barrier 126.

The absorbent article 100 may include waist elastics in (a) the front waist region 102; (b) the rear waist region 104; or (c) both the front waist region 102 and the rear waist region 104. Elasticity may be provided to the front waist region 102 and/or the rear waist region 104 by any means known in the art. For example, elastic threads may be adhered directly to the backsheet 114 and/or the topsheet 112 and may gather the front waist region 102 and/or rear waist region 104. In a second example, the topsheet 112 and/or backsheet 114 may be formed from an elastic material. In a third example, as shown in FIGS. 2A and 2B, an elastic waistband 144 may be provided within at least one of the front waist region 102 and/or the rear waist region 104. It is to be understood that elasticity may be provided by a first means (e.g., elastic threads) to the front waist region and may be provided to the rear waist region by a second means (e.g., elastic film) which is different from the first means.

In the example shown in FIGS. 2A and 2B, an elastic waistband 144 is provided in the rear waist region 104 to provide elasticity thereto. The elastic waistband 144 may be formed from any material or combination of materials known in the art for providing elasticity to the waist region. For example, the elastic waistband may include an elastic film or a plurality of elastic threads.

Any portion of the front waist region 102 and/or rear waist region 104 may be elasticized. Optionally, the elastic portion of the front waist region 102 and/or the rear waist 104 region may be positioned outboard the absorbent barrier 126. That is, the absorbent barrier 126 may or may not overlap with an elastic portion of the front waist region 102 and/or the rear waist region 104. That is, in exemplary absorbent articles 100 that include an absorbent barrier 126 inset from the front waist edge 108 (as shown in FIG. 2B) and waist elastics, the waist elastics may extend (a) solely within the zone between the front barrier edge 130 and the front waist edge 108; or (b) within the zone between the front barrier edge 130 and the front waist edge 108 and overlapping with the absorbent barrier 126.

It may be desirable for the absorbent article 100 to include waist elastics outboard the absorbent barrier 126 to improve comfort when wearing the absorbent article 100. In examples where absorbent article 100 include waist elastics and where the absorbent barrier 126 extends to the waist edge 108, 110, the absorbent barrier 126 may limit the amount the waist elastics may gather the topsheet 112 and/or the backsheet 114.

With reference to FIG. 3B, and as stated above, the absorbent article 100 may include an absorbent barrier 126 positioned at the rear core edge 120. The absorbent barrier when positioned at the rear core edge 120 may have all the same characteristics of the absorbent barrier positioned at the front core edge 118. That is, for example, as shown in FIG. 3B, the absorbent barrier 126 may be positioned such that the front barrier edge 130 is positioned longitudinally forward the rear core edge 120 and the rear barrier edge 132 is positioned longitudinally rearward the rear core edge 120. Optionally, as shown, the rear barrier edge 132 may be positioned longitudinally between the rear core edge 120 and the rear waist edge 110. Alternatively, the rear barrier edge 132 may extend to the rear waist edge 110.

The position of the absorbent barrier 126 relative to the front or rear core edge 118, 120 may vary depending on the size of the absorbent article 100. That is, the absorbent barrier 126 may have a longitudinal center, and that longitudinal center may be positioned forward, rearward, or aligned with the front or rear core edge 118, 120. In the example illustrated in FIG. 1B, the longitudinal center of the absorbent barrier 126 is aligned with the front core edge 118. Likewise, in the example illustrated in FIG. 3B, the longitudinal center of the absorbent barrier 126 is aligned with the rear core edge 120. That is, in the example shown in FIG. 1B, the front barrier edge 130 is spaced from the front core edge 118 by a first distance 150, the rear barrier edge 132 is spaced from the front core edge 118 by a second distance 152, and the first distance 150 is equal to the second distance 152 (i.e., the front barrier edge 130 and the rear barrier edge 132 may be longitudinally equally spaced from the front core edge 118).

In addition to the longitudinal center of the absorbent barrier 126 being offset or aligned with the front or rear core edge 118, 120, the longitudinal center of the absorbent core 116 may be offset or aligned with the longitudinal center of the absorbent article 100. That is, the front core edge 118 may be longitudinally spaced from the front waist edge 108 by a first distance 154 (as shown in FIG. 1B), the rear core edge 120 may be longitudinally spaced from the rear waist edge 110 by a second distance 156, and the first distance 154 may be equal to, less than, or greater than the second distance 156. In the example illustrated, the first distance 154 is less than the second distance 156. Optionally, the first distance 154 may be at least 20 mm less than the second distance 156.

Still referring to FIGS. 1A and 1B, the portion of the absorbent barrier 126 that overlaps with the absorbent core 116 may be positioned between the absorbent core 116 and the topsheet 112 in the thickness direction. It has been found that positioning a portion of the absorbent barrier 126 above the absorbent core 116 in the thickness direction may increase the likeliness of the absorbent barrier 126 capturing any bodily fluid deposited into the absorbent article 100 not absorbed by the absorbent core 116. While the absorbent barrier 126 is illustrated in FIG. 1B as being planar in the thickness direction, it will be appreciated that when the absorbent article 100 is assembled, a portion of the absorbent barrier 126 may be aligned with a portion of the absorbent core 116 in the thickness direction. Accordingly, the thickness of the absorbent article 100 where the absorbent barrier 126 overlaps the absorbent core 116 may be greater than that of where the absorbent barrier 126 does not overlap the absorbent core 116.

In the example shown in FIGS. 1A and 1B, the absorbent barrier 126 directly abuts the absorbent core 116. That is, there are no layers of material (e.g., topsheet 112) between the absorbent barrier 126 and the absorbent core 116. It has been determined that directly abutting the absorbent barrier 126 to the absorbent core 116 may increase the likeliness of the absorbent barrier 126 capturing any urine not absorbed by the absorbent core 116.

In some examples, the absorbent barrier 126 may be directly secured to the absorbent core 116 (e.g., by an adhesive). In other examples, the absorbent barrier 126 may not be directly secured to the absorbent core 116. When the absorbent barrier 126 is not directly secured to the absorbent core 116, the absorbent barrier 126 may be directly secured to the topsheet 112, the absorbent core 116 may be directly secured to the backsheet 114, and the topsheet 112 may be directly secured to the backsheet 114 to hold the absorbent barrier 126 in a predetermined position relative to the absorbent core 116. It may be desirable to not directly secure the absorbent barrier 126 to the absorbent core 116 as adhesive or other bonding agents may limit the absorption capabilities of the absorbent barrier 126 and/or the absorbent core 116. Further, any adhesive or bonding agent positioned between the absorbent barrier 126 and the absorbent core 116 may inhibit transfer of fluid between the absorbent barrier 126 and the absorbent core 116.

Referring now to FIGS. 4A to 4C, the absorbent article 100 may include a first longitudinally extending barrier leg cuff 160 and a second longitudinally extending barrier leg cuff 162. As shown, the each of the barrier leg cuffs 160, 162 may include a barrier web 164 and one or more elastic strands 166. In the example illustrated, the barrier leg cuffs 160, 162 are coupled to the topsheet 112. In other examples the barrier leg cuffs 160, 162 may be integrally formed with the topsheet 112. As will be understood by a person of skill in the art, the barrier leg cuffs 160, 162 may cooperate to form a lateral barrier.

In the example illustrated in FIG. 4C, each of the barrier leg cuffs 160, 162 include a standing cuff portion 168 that extends upwardly from the topsheet 112 to form the lateral barrier. Depending on the size of the absorbent article 100, the distance 170 between the standing cuff portions 168 of the barrier leg cuffs 160, 162 (herein referred to as the lateral distance 170 between the barrier leg cuffs 160, 162) may vary. It is to be understood that barrier leg cuffs 160, 162 that are integrally formed with the topsheet 112 may also include a standing cuff portion 168.

In some embodiments, as shown in FIG. 4C, the lateral distance 170 between the barrier leg cuffs 160, 162 may be less than the lateral width 140 of the absorbent barrier 126. It may be desirable for the absorbent barrier 126 to have a lateral width 140 greater than the lateral distance 170 between the barrier leg cuffs 160, 162 so that any liquid that runs along the crevice created by the respective barrier leg cuff 160, 162 and the topsheet 112 may be directed to and absorbed by the absorbent barrier 126.

Still referring to FIGS. 4A to 4C, the absorbent article 100 may further include an acquisition layer 172. The acquisition layer 172 may more readily absorb and distribute liquids than the absorbent core 116. The acquisition layer 172 may be made of any material and may be of any shape and size known in the art. In some examples, the acquisition layer 172 may be made of a high loft carded nonwoven. In other examples, the acquisition layer 172 may include or be formed of an apertured poly film. The apertures of the apertured poly film may be 3-dimensional.

As shown in FIGS. 4A to 4C, the acquisition layer 172 may be positioned such that it (a) overlaps the absorbent core 116; and (b) does not overlap the absorbent barrier(s) 126. While the example shown illustrates the acquisition layer 172 being longitudinally spaced apart from the absorbent barrier(s) 126, in other examples, the acquisition layer 172 may abut the absorbent barrier(s) 126. In yet another example, the acquisition layer 172 may overlap each of the absorbent core 116 and the absorbent barrier(s) 126. It is to be understood that an absorbent article 100 may include only one absorbent barrier 126 positioned at one of the front core edge 118 and the rear core edge 120 of the absorbent core 116 and an acquisition layer 172.

As noted above, the absorbent barrier 126 may not be directly secured to the absorbent core 116. Accordingly, to manufacture the absorbent article 100 as shown in FIGS. 1A to 4C, the following steps may be performed:
providing a topsheet 112, a backsheet 114, an absorbent core 116, and an absorbent barrier 126;
securing the absorbent barrier 126 to the topsheet 112;
securing the absorbent core 116 to the backsheet 114;
positioning the absorbent barrier 126 so that a first portion of the absorbent barrier 126 overlaps with the absorbent core 116 in a thickness direction and a second portion of the absorbent barrier 126 does not overlap with the absorbent core 116 in the thickness direction; and
securing the topsheet 112 to the backsheet 114.

It is to be understood that the steps described above may be performed in an alternative order to what is described above. That is, for example, the absorbent core 116 may be secured to the backsheet 114 before or after the absorbent barrier 126 is secured to the topsheet 112.

When manufacturing the absorbent article 100 as shown in FIGS. 1A to 4C, the method may include the steps of providing elasticity to at least one of the front waist region 102 and the rear waist region 104. In some exemplary methods, providing elasticity to at least one of the front waist region 102 and the rear waist region 104 may include providing an elastic waistband 144 and securing the elastic waistband 144 to the topsheet 112 and/or the backsheet 114. Optionally, when elasticity is to be provided to each of the front waist region 102 and the rear waist region 104, a second waistband 144 may be provided. Like the first waistband 144, the second waistband 144 may be secured to the topsheet 112 and/or backsheet 114. Specifically, the first elastic waistband may be secured to the topsheet 112 and/or the backsheet 114 in the front waist region 102 and the second elastic waistband 144 may be secured to the topsheet 112 and/or the backsheet 114 in the rear waist region 104.

When manufacturing the absorbent article 100 as shown in FIGS. 1A to 4C, the method may include the steps of providing a second absorbent barrier 126 and securing the second absorbent barrier 126 to the topsheet 112. Prior to securing to second absorbent barrier 126 to the topsheet 112, the absorbent barrier 126 may be positioned so that a first portion of the second absorbent barrier 126 overlaps with the absorbent core 116 in a thickness direction and a second portion of the second absorbent barrier 126 does not overlap with the absorbent core 116 in the thickness direction. Optionally, the absorbent barrier 126 may be secured to the topsheet 112 prior to the positioning step. In accordance with the example shown in FIGS. 4A to 4C, the absorbent core 116 may have a first distal end and a second distal end and the first absorbent barrier 126 may be positioned at the first distal end of the absorbent core 116 (i.e., proximate front core edge 118) and the second absorbent barrier 126 may be positioned at the second distal end of the absorbent core (i.e., proximate rear core edge 120).

When manufacturing the absorbent article 100 as shown in FIGS. 1A to 4C, the method may include the step of directly abutting the absorbent core 116 to the absorbent barrier 126. It is noted that the step of directly abutting the absorbent core 116 to the absorbent barrier may not include a step of directly securing the absorbent core 116 to the absorbent barrier 126. As noted above, the absorbent core 116 may not be directly secured to the absorbent barrier 126. Further, it is to be understood that the absorbent article 100 may include multiple absorbent barriers 126 and each of the absorbent barriers 126 may directly abut the absorbent core 116.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims is not limited to the examples set out herein, but should be understood in a manner consistent with the description as a whole.

We claim:

1. An absorbent article comprising:
a front waist region having front waist edge, a rear waist region having a rear waist edge, and a crotch region extending longitudinally between the front waist region and the rear waist region;
a liquid pervious topsheet on a skin facing side of the absorbent article;
a liquid impervious backsheet on a garment facing side of the absorbent article;
an absorbent core positioned between the topsheet and the backsheet in a thickness direction, the absorbent core having a front core edge a rear core edge and a pair of side core edges extending between the front core edge and the rear core edge; and
an absorbent barrier, the absorbent barrier having a front barrier edge, a rear barrier edge, and a pair of side barrier edges extending between the front barrier edge and the rear barrier edge,
the front barrier edge positioned longitudinally forward the front core edge,
the rear barrier edge positioned longitudinally rearward the front core edge, and
the rear barrier edge positioned between the absorbent core and the topsheet in the thickness direction.

2. The absorbent article of claim 1, wherein
the front barrier edge is positioned longitudinally between the front core edge and the front waist edge, and
the rear barrier edge is positioned longitudinally between the front core edge and the rear core edge.

3. The absorbent article of claim 1, wherein
the front core edge is longitudinally spaced from the front waist edge by a first distance,
the rear core edge is longitudinally spaced from the rear waist edge by a second distance, and
the first distance is greater than the second distance.

4. The absorbent article of claim 3, wherein the first distance is at least 20 mm greater than the second distance.

5. The absorbent article of claim 1, wherein absorbent barrier directly abuts the absorbent core.

6. The absorbent article of claim 1, wherein the absorbent barrier is secured to the topsheet and the absorbent core is secured to the backsheet.

7. The absorbent article of claim 1, wherein the front barrier edge and the rear barrier edge are longitudinally equally spaced from the front core edge.

8. The absorbent article of claim 1, wherein the front barrier edge is longitudinally spaced between 20 mm and 30 mm from front waist edge.

9. The absorbent article of claim 1, further comprising a first longitudinally extending barrier leg cuff and a second longitudinally extending barrier leg cuff, the first barrier leg cuff and the second barrier leg cuff laterally spaced apart by a third distance,
wherein
the absorbent barrier has a lateral width; and
the lateral width of the absorbent barrier is greater than the third distance.

10. The absorbent article of claim 1, further comprising an acquisition layer positioned between the topsheet and the absorbent core in the thickness direction, the acquisition layer having a front acquisition edge and a rear acquisition edge, each of the front acquisition edge and the rear acquisition edge positioned longitudinally rearward rear barrier edge.

11. The absorbent article of claim 1, wherein the absorbent barrier is formed from an airlaid material.

12. The absorbent article of claim 1 further comprising a second absorbent barrier, the second absorbent barrier having a front barrier edge, a rear barrier edge, and a pair of side barrier edges,
the front barrier edge of the second absorbent barrier positioned longitudinally forward the rear core edge,
the rear barrier edge of the second absorbent barrier positioned longitudinally rearward the rear core edge, and
the front barrier edge of the second absorbent barrier positioned between the absorbent core and the topsheet in the thickness direction.

13. The absorbent article of claim 12, wherein
the front barrier edge of the second absorbent barrier is positioned longitudinally between the rear core edge and the front core edge, and
the rear barrier edge of the second absorbent barrier is positioned longitudinally between the rear core edge and the rear waist edge.

14. The absorbent article of claim 12 wherein the front barrier edge of the second absorbent barrier and the rear barrier edge of the second absorbent barrier are longitudinally equally spaced from the rear core edge.

15. The absorbent article of claim 12, wherein the rear barrier edge of the absorbent barrier and the front barrier edge of the second absorbent barrier are longitudinally spaced apart.

16. An absorbent article comprising:
a front waist region having front waist edge, a rear waist region having a rear waist edge, and a crotch region extending longitudinally between the front waist region and the rear waist region,
a liquid pervious topsheet on a skin facing side of the absorbent article;
a liquid impervious backsheet on a garment facing side of the absorbent article;
an absorbent core positioned between the topsheet and the backsheet in a thickness direction, the absorbent core having a front core edge a rear core edge and a pair of side core edges extending between the front core edge and the rear core edge; and
an absorbent barrier, the absorbent barrier having a front barrier edge, a rear barrier edge, and a pair of side barrier edges extending between the front barrier edge and the rear barrier edge,
the front barrier edge positioned longitudinally forward the rear core edge,
the rear barrier edge positioned longitudinally rearward the rear core edge, and
the front barrier edge positioned between the absorbent core and the topsheet in the thickness direction.

17. The absorbent article of claim 14, wherein
the rear barrier edge is positioned longitudinally between the rear core edge and the rear waist edge, and
the front barrier edge is positioned longitudinally between the front core edge and the rear core edge.

18. The absorbent article of claim 16, wherein the front barrier edge and the rear barrier edge are longitudinally equally spaced from the rear core edge.

19. A method of manufacturing an absorbent article comprising:
providing a topsheet, a backsheet, an absorbent core, and an absorbent barrier;
securing the absorbent barrier to the topsheet;
securing the absorbent core to the backsheet;
positioning the absorbent barrier so that a first portion of the absorbent barrier overlaps with the absorbent core in a thickness direction and a second portion of the absorbent barrier does not overlap with the absorbent core in the thickness direction; and
securing the topsheet to the backsheet.

20. The method of claim 19, wherein the method further comprises:
providing an elastic waist band; and
securing the elastic waistband to at least one of the topsheet and the backsheet.

21. The method of claim 19, wherein the method further comprises:
providing a second absorbent barrier;
securing the second absorbent barrier to the topsheet; and
positioning the second absorbent barrier so that a first portion of the second absorbent barrier overlaps with the absorbent core in a thickness direction and a second portion of the second absorbent barrier does not overlap with the absorbent core in the thickness direction,
wherein the absorbent core has a first distal end and a second distal end and the absorbent barrier is positioned at the first distal end of the absorbent core and the second absorbent barrier is positioned at the second distal end of the absorbent core.

22. The method of claim 19, wherein the method further comprises:
providing a second elastic waist band; and
securing the second elastic waist band to at least one of the topsheet and the backsheet;
wherein the absorbent article has a front waist region and a rear waist region and the elastic waist band is secured to the at least one of the topsheet and the backsheet in the front waist region and the second elastic waist band is secured to the at least one of the topsheet and the backsheet in the rear waist region.

23. The method of claim 19, wherein the method further comprises directly abutting the absorbent barrier with the absorbent core.
